(19) United States
(12) Patent Application Publication
(10) Pub. No.: US 2021/0000621 A1
(43) Pub. Date: Jan. 7, 2021

(54) PROSTHETIC LIMB SLEEVE

(71) Applicant: ARTEMUS LABS, Long Beach, CA (US)

(72) Inventors: Calvin Leung, Garden Grove, CA (US); Nicole Escobar, Bellflower, CA (US); Rae Jillian Ramiscal Rivera, Long Beach, CA (US); Kimberly Antonio Ventura, Long Beach, CA (US)

(21) Appl. No.: 16/915,861

(22) Filed: Jun. 29, 2020

Related U.S. Application Data
(60) Provisional application No. 62/869,917, filed on Jul. 2, 2019.

(51) Int. Cl. A61F 2/78; B32B 27/40

(52) U.S. Cl. CPC A61F 2/7812; A61F 2002/785; A61F 2002/7837; B32B 27/40

(57) ABSTRACT

A sleeve for mounting a prosthetic unit on a limb is formed using a tripartite composite that includes a breathable woven fabric in combination with an elastic urethane middle layer and a soft perforated plastic inner layer. The middle layer can be a hyper-elastic polymer that is cured to a hemispherical shape to stretch and move while maintaining a shape memory that returns the polymer to its original shape once relieved of a stretching force. Moreover, the liner/prosthetic sleeve of the present invention's anatomical suspension works as an advantage to maintain its placement on the user's residual limb.

### CROSS-REFERENCES TO RELATED APPLICATIONS

This application claims priority from U.S. Provisional Patent Application No. 62/869,917, filed Jul. 2, 2019, the content of which is incorporated by reference herein in its entirety.

### BACKGROUND

The invention relates to prosthetic coverings, and more specifically to a prosthetic sleeve that is breathable and more comfortable than existing coverings. This invention relates to artificial limbs, and particularly to the attachment of prosthetic units to limbs. Attachment devices for prosthetics typically use a textile or other gelatinous material such as silicone, polyurethane or copolymer sleeve adapted to overlay the residual portion of the limb, at the end of which a mechanism for coupling to the respective prosthetic. Such arrangements are disclosed in U.S. Pat. No. 6,231,617; US Patent publication Nos. 2005/0240283 and 2002/0183859; and International Patent publication Nos. WO 00/51531 and WO 00/51537. The disclosures of all of these documents are hereby incorporated by reference.

Prosthetic legs or arms, for example, are fitted to the residual limb of an amputee by using a flexible sleeve/liner. Current prosthetic sleeves are conventionally constructed from textile fabrics coated with an impermeable sheet of silicone rubber or other gelatinous polymers. Consequently, heat is then trapped causing users to sweat which is not transported away from the skin and accumulates within the sleeves. This moisture can cause bacterial which results in odor, rash, and other issues that pose problems for the wearer. To be comfortable, a sleeve/liner for use in attaching a prosthetic unit to a residual limb should not only facilitate the movement of moisture and heat from the skin, but also minimize the movement of the sleeve relative to the limb without applying undue pressure on the flesh against which it is held. In addition, the resting temperature of the residual limb must be maintained as much as possible by not entrapping heat and allowing for airflow to facilitate cooling. The present invention seeks to address these issues and provide a sleeve which meets these objectives.

### SUMMARY OF THE INVENTION

According to the invention, a sleeve for mounting a prosthetic unit on a limb is formed using a tripartite composite forming a breathable woven fabric that can be used to cover a limb and used in conjunction with a prosthetic device. The outer layer is a breathable elastic fabric formed with holes in a spaced apart relationship to introduce airflow into the sleeve. An intermediate layer is formed of a flexible, thermoplastic polymer that can expand and form a cushion for the wearer. The innermost layer is preferably a soft, perforated plastic, and each layer is sealed to form a single composite material. The elastomer can be cured to a hemispherical or conical shape to stretch and move while retaining a shape memory that returns the polymer to its original shape once relieved of a stretching force. Moreover, the liner/prosthetic sleeve or cone of the present invention's anatomical suspension works as an advantage to maintain its placement on the user's residual limb. These and other features of the invention will best be understood with reference to the drawings and the detailed description of the invention below.

### BRIEF DESCRIPTION OF THE DRAWINGS

FIG. 1 is an elevated, perspective view of a first embodiment of the present invention;

FIG. 2 is an enlarged, perspective view in shadow of the sleeve of the present invention;

FIG. 3 is an enlarged, sectional view of the tripartite layers of the present invention;

### DETAILED DESCRIPTION OF THE PREFERRED EMBODIMENTS

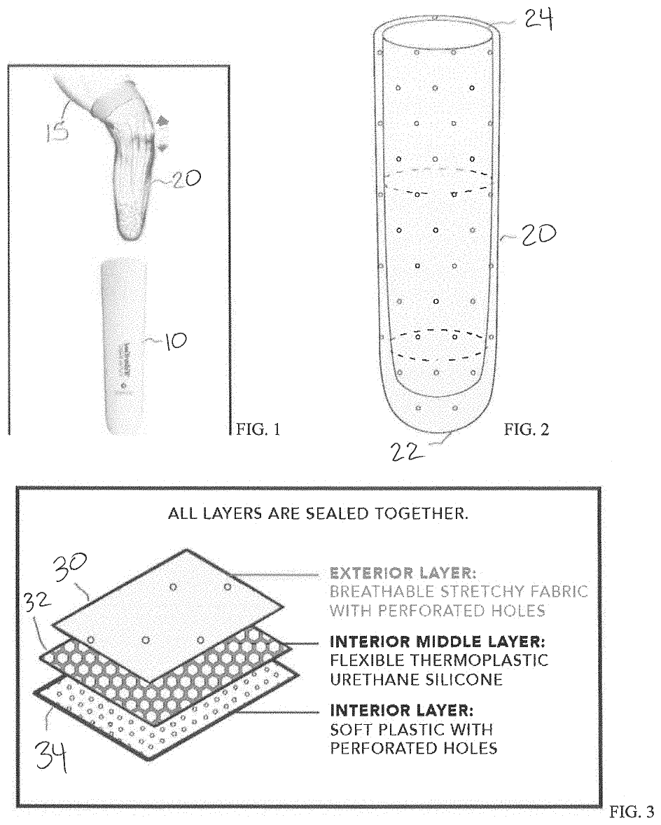

FIG. 1 illustrates how an amputated leg fits inside of a prosthetic device. The prosthetic device 10 is hollow and the limb 15 is covered with a sleeve 20 that protects the limb from wear, friction, abrasion, and general discomfort from wearing the prosthetic for long periods of time. Such sleeves 20 are typically made of a soft material such as a sock that can easily be slipped over the limb, or a more protective material that offers more protection at the expense of comfort and breathability. The present invention offers both comfort and protection in a unique tripartite material composition. This design also allows the sleeve to become more breathable and to facilitate airflow from hot spots, with the hyper-elastic polymer material adding a waterproof and hygienic feature. The sleeve has the capability to support highly advanced fabrics to provide a supportive, breathable mesh on the inner and outer shell of the prosthetic liner.

FIG. 2 illustrates a hollow cylindrical sleeve 20 having a closed first end 22 and an open second end 24. The sleeve is sized to fit an amputated limb with some snugness, so it is designed to stretch around the limb for a close but not tight fit. The sleeve 20 has spaced apart holes over the entire surface of the sleeve for aerating the limb and for cooling purposes. In a first embodiment, the grooves are recessed and lead to air channels that line the inside wall. With this, air and moisture is able to travel both horizontally and vertically to escape through the outer surface. Channels and pathways built on the inner wall of the liner facilitate the movement, flow and circulation of air and moisture to cool the residual limb as well as allowing for hot air to escape. This allows for the liner to passively maintain and cool the residual limb as the user wears it.

FIG. 3 shows the three layers that make up the composition of the composite sleeve 20 of the present invention. The outer layer 30 is a breathable, elastic fabric that incorporates holes at regular intervals over the surface of the material. The outer layer 30 must be of a nature that it can bond with the intermediate layer, either through thermal bonding or some other bonding technique. The middle layer 32 is a flexible thermoplastic urethane silicone that forms a honeycomb structure with openings that permit moisture and heat to pass through. The flexible thermoplastic urethane silicone is stretchable in all directions and is sandwiched between the outer and inner layers. The inner layer 34 is a soft plastic material with perforations and has a thickness that is approximate to the middle layer 32. The inner layer is moisture wicking to extract perspiration and wick it to the outer layer while providing a soft and more comfortable interface with the patient's skin. The three layers are sealed together into a unitary composite and formed into the cylindrical or conical sleeve 20 as shown in FIG. 2.

The sleeve 20 of the present invention can be manufactured in multiple ways depending on the material used at the time of production. One manufacturing process involves the tooling of negative molds that are lined with the selected moisture wicking fabric to be joined with a urethane silicone polymer that is poured in and cured to the specified shape and design. There are multiple molds based on thickness and sizing to fit an array of individuals. These molds include the recessed air channels and pathways along with the holes to allow for additional air flow. Another method uses a prefabricated thermoformable cone made from a foam like material. After selecting and adjusting the material to a predetermined thickness, length and size, it is placed on a positive mold where it is then covered and placed in a vacuum forming and heating machine. After approximately 5-10 minutes it is removed and allowed to cool. The mold includes the positive indentations that emboss the design on the inner walls of the liner. The final step for both manufacturing process involves inspecting for imperfections, quality testing, and final adjustments to ensure efficacy.

While specific embodiments of the present invention have been described herein, the invention is not limited to the embodiments shown or described but rather the breadth of the invention includes non-described embodiments. A person of ordinary skill in the art would readily recognize various modifications, substitutions, and alterations to the above described embodiments, and the scope of the invention is intended to include all such modifications, substitutions, and alterations.

We claim:

1. A sleeve for use in conjunction with mounting a prosthetic device on a limb, comprising:
a hollow cylindrical sleeve having a closed first end and an open second end, the cylindrical sleeve formed of a unitary tripartite composite, comprising:
an outer layer of a breathable elastic fabric formed with spaced apart holes;
an intermediate layer formed in a honeycomb pattern of a flexible, thermoplastic polymer; and
an innermost layer of a perforated plastic;
wherein each of the layers are sealed together to form a unitary composite.

2. The sleeve of claim 1, wherein the intermediate layer is formed of a flexible thermoplastic urethane silicone.

3. The sleeve of claim 2, wherein a thickness of the intermediate layer is equal to a thickness of the innermost layer.
